(19) United States
(12) Patent Application Publication
(10) Pub. No.: US 2019/0262164 A1
(43) Pub. Date: Aug. 29, 2019

(54) WELDED ORHOPEDIC ANKLE SUPPORT FOR SELECTIVELY STABILIZING ANKLE MOVEMENT AND METHOD FOR MAKING SAME

(71) Applicant: Ortho Systems d/b/a Ovation Medical, Agoura Hills, CA (US)

(72) Inventors: Kenji Watabe, Ventura, CA (US); Tracy E. Grim, Thousand Oaks, CA (US); Edwin Erwin, Studio City, CA (US)

(21) Appl. No.: 15/905,747

(22) Filed: Feb. 26, 2018

(51) Int. Cl. A61F 5/058; A61F 5/01; A61L 15/14; A61L 15/12; B29C 65/70; B29C 65/00

(52) U.S. Cl. CPC A61F 5/0585; A61F 5/05825; A61F 5/0111; A61L 15/14; B29K 2075/00; B29C 65/70; B29C 66/436; B29C 66/112; A61L 15/12

(57) ABSTRACT

An orthopedic ankle support is disclosed which provides an ultra-thin profile and selective restriction of movement of the user's ankle. In an embodiment, sections of a thin, tense material are welded onto a non-rigid body. The sections may include a tense anchor segment which may be proximate the heel opening and/or sole, and one or more tension segments that may extend from the anchor segment to upper portions of the support. In an embodiment, certain of the tension segments may be connected to an attachment portion affixed on the body, such as a thick lace-up area for tightening the support. Welding of the tension segments onto the body using thermal fusion obviates the need for uncomfortable stitching and material lumps. In addition, welding the tension segments into predetermined orientations enables the designer to have control over the properties of the support such that, for example, the tension segments may be oriented so as to restrict undesirable rotatory motion of the ankle without restricting natural forward and rearward motions of the foot.

### BACKGROUND

### Field

The present disclosure relates generally to anatomical supports, and more particularly, to a low profile orthopedic ankle support for reducing edema and selectively restricting movement of a user's ankle.

### Background

Acute ankle injuries are the most common acute sport trauma, accounting for about fourteen (14) percent of all sports related injuries. Among eighty (80) percent are ligamentous sprains caused by explosive inversion or supination. The etiology of most ankle sprains is due to incorrect foot positioning at landing during activity. The ligamentous structures are exposed to sudden high explosive forces in an extremely short amount of time, resulting in grade 1, 2, or 3 ligamentous injuries and fractures in and about the joint. In short, the offending moments are presented and actuated before our body can compensate.

Ligaments are generally strong elements designed to facilitate proper joint operation by inhibiting a joint from moving in a direction in which the joint is not designed to move. Numerous ligaments and ligament types are associated with various portions of the human anatomy. The ankle joint, for one, is associated with certain well-known ligaments. When an injury occurs such that one or more ligaments are torn, the remaining ligaments are left to bear the resistance resulting from any ankle movement in order to maintain proper operation of the joint while the injury heals. Sprains, strains and other injuries involving ligaments and ankle tissue are painful, can cause considerable swelling and can endure for some time.

A number of orthopedic supports are currently available to assist in healing for people with ankle injuries including sprains, torn ligaments, and the like. For individuals suffering from severe ankle injuries such as those involving multiple ligaments or bone fractures, a cast or walking boot may be suitable. For the majority of individuals whose sprains and strains are less intense in magnitude, other options may be available. These include primarily non-rigid ankle supports that offer greater flexibility. In principle, these more compact and more flexible supports allow the user to move around, e.g., to walk, with the support in place. Many current braces are designed to be worn with a shoe over the brace.

The archetypical support in this category may include a body defined by various materials, such as, in certain commercial supports, a tense, canvas-like material encompassing the entire body or a substantial portion of the body and a lace-up portion or other tightening mechanism. The uniformly-disposed tense material in these supports is designed to tightly encompass the foot to accent venous flow of blood up the leg and thereby reduce swelling (which in turn reduces pain) and, equally importantly, to restrict the unwanted ankle rotation that led to the injury in the first instance.

Because it can take six weeks or longer for ankle injuries of these types to heal, a pivotal consideration of any such ankle support is comfort. An uncomfortable brace will, more often than not, motivate the user to avoid use of the brace as often as instructed. This consequence can lead to longer healing times. Unfortunately, conventional commercial ankle braces typically involve often multiple layers of material stitched together, rendering these available commercial braces unnecessarily thick and unwieldy. The materials used can also be rough to the touch. They also include protrusions running along lateral portions of the brace due to the stitching. These protrusions tend to dig into the sensitive areas of the foot over time, causing added discomfort. The material may also be designed to include a significant amount of tension to enable the support, when worn properly, to reduce edema (swelling) and to restrict ankle and foot movement. The materials used in conventional braces, often stitched together in layers as described above, lack breathability, which can further irritate the skin. Also, the thick, high tension material is included in many conventional braces below the foot. This can be particularly uncomfortable to a user when walking or standing for extended periods of time, particularly with footwear over the brace. Thus, especially for areas below the shoeline, the thickness of these conventional devices can be a significant problem because they cause discomfort that is likely to worsen over the duration of the injury period. All of the aforedescribed problems reduce the overall motivation for the user to wear the support on a consistent basis to allow the ankle to heal in as short a time as possible.

Another problem with existing ankle supports is that, with their presence of high tension fabric disposed substantially over the entire body of the support to support the ankle, even the lowest-profile (thinnest) available commercial braces fail to discriminate in their restrictions to the foot/ankle system on motion. That is, with these conventional braces, foot motion is limited or restricted in all directions. These braces may resemble a bootie with a heel and a toe opening. Substantially the entire surface of the support, including the rear portion encompassing the sole of a user's foot, may be composed of an often thick and/or very tense material that, when donned by a user, offers the user little flexibility to move the user's foot even in directions necessary for the user to comfortably walk forward. In short, the conventional brace not only restricts unwanted rotation of the ankle that caused the injury, but also restricts natural forward and backward movement of the foot that allows a user to comfortably walk.

These and other deficiencies are addressed in the present disclosure.

### SUMMARY

Several aspects of an ultra-low-profile motion-selective orthopedic ankle support and method for producing same are disclosed.

In an aspect of the disclosure, an orthopedic ankle support includes a non-rigid body and one or more sections of tense material welded along at least portions of one or both sides of the body and configured to inhibit ankle pronation and supination while promoting dorsi and plantar flexion.

In another aspect of the disclosure, an orthopedic ankle support includes a bootie, one or more tension segments welded to the bootie to at least partially enclose a bootie heel region and to extend radially from the bootie heel region along at least one side of the bootie, wherein the one or more tension segments are configured to inhibit rotatory ankle motion while promoting dorsi and plantar flexion.

In another aspect of the disclosure, an orthopedic ankle support includes a non-rigid body having an open heel region, the body including an anchor segment, and at least one tension segment connected to the anchor segment and extending across at least a portion of at least one side of the body, wherein the at least one tension segment is welded to the body and configured to provide directional support for selectively stabilizing the ankle.

In another aspect of the disclosure, an orthopedic ankle support includes a non-rigid body having an open heel region, the body including at least one tension segment extending across at least a portion of at least one side of the body, wherein the at least one tension segment is welded to the body and configured to provide directional support for selectively stabilizing the ankle.

In another aspect of the disclosure, an orthopedic ankle support includes a non-rigid body having an open heel region, the body including two or more pieces of material welded together to form a bootie, wherein at least one welded region of the bootie is oriented to provide directional support to selectively stabilize the ankle, and wherein the at least one welded region comprises sufficient tensile strength to provide the directional support.

In another aspect of the disclosure, a method for making an orthopedic ankle support includes welding together two or more pieces of material to form a bootie; and orienting at least one welded region of the bootie to provide directional support to selectively stabilize the ankle, wherein the oriented at least one welded region comprises tensile strength sufficient to provide the directional support.

In another aspect of the disclosure, an orthopedic ankle support includes a non-rigid body having open toe and heel regions, the body including an attachment portion along opposing edges of the body at a front of the foot; an anchor segment extending along at least a portion of one or both of (i) an area proximate the open heel region and (ii) a sole region; at least one tension segment extending from the anchor segment to the attachment portion on at least one side of the body; wherein one or both of the anchor and tension segments are welded to the body and configured to provide directional tension to inhibit rotatory ankle motion while promoting dorsi and plantar flexion.

In another aspect of the disclosure, a method for producing an orthopedic ankle support comprising a non-rigid body configured to provide directional support for selectively stabilizing the ankle, aligning flat tension sections with a fabric body to produce a stack of aligned materials, the aligned materials of the stack being aligned directly or via a substrate material configured to facilitate thermal fusion, applying heat and pressure only to predetermined portions of the stack, including the flat tension sections, to weld the predetermined portion leaving the remaining stack portions unwelded, and shaping the resulting stack to form the ankle support.

It is understood that other aspects will become readily apparent to those skilled in the art from the following detailed description, wherein it is shown and described only exemplary configurations of an orthopedic ankle support by way of illustration. As will be realized, the present disclosure includes other and different aspects and its several details are capable of modification in various other respects, all without departing from the spirit and scope of the present disclosure. Accordingly, the drawings and the detailed description are to be regarded as illustrative in nature and not as restrictive.

### BRIEF DESCRIPTION OF THE DRAWINGS

Various aspects of the present invention are illustrated by way of example, and not by way of limitation, in the accompanying drawings, wherein:

FIG. 1A is a front perspective view illustrating an example of an orthopedic ankle support according to the present disclosure.

FIG. 1B is a rear perspective view illustrating an example of an orthopedic ankle support according to the present disclosure.

FIG. 1C is a side perspective view illustrating an example of an orthopedic ankle support according to the present disclosure.

FIG. 1D is a rear perspective view illustrating an example of an orthopedic ankle support according to the present disclosure.

FIG. 2A is a side view illustrating the bootie of an orthopedic ankle support according to the disclosure.

FIG. 2B is a front view illustrating the bootie of FIG. 2A.

FIG. 2C is a rear view illustrating the bootie of FIG. 2A.

FIG. 3A is a side view illustrating one embodiment of the tension segments of the orthopedic support of the present disclosure.

FIG. 3B is a side view illustrating another embodiment of the tension segments of the orthopedic support of the present disclosure.

FIG. 4A is a side view illustrating another embodiment of the tension segments of the orthopedic support of the present disclosure.

FIG. 4B is a side view illustrating another embodiment of the tension segments of the orthopedic support of the present disclosure.

FIG. 5A is a side view illustrating another embodiment of the tension segments of the orthopedic support of the present disclosure.

FIG. 5B is a side view illustrating another embodiment of the tension segments of the orthopedic support of the present disclosure.

FIG. 6A is a side perspective view illustrating an embodiment of the orthopedic ankle support with a figure eight structure attached.

FIG. 6B is another side perspective view illustrating an embodiment of the orthopedic ankle support with a figure eight structure attached.

FIG. 7 illustrates an exemplary pair of graphs of stress versus strain and the resulting value for Young's modulus for different materials.

FIG. 8 is a perspective exploded view illustrating an exemplary layering of materials for constructing the body of the orthopedic ankle support.

FIG. 9 is a flow diagram illustrating an exemplary method for producing an orthopedic ankle support according to the disclosure.

### DETAILED DESCRIPTION

Various aspects of an orthopedic ankle support will now be presented. However, as those skilled in the art will readily appreciate, these aspects may be extended to other anatomical supports without departing from the spirit and scope of the present disclosure. The detailed description set forth below in connection with the appended drawings is intended to provide a description of various exemplary embodiments of techniques for an orthopedic ankle support and is not intended to represent the only embodiments in which the invention may be practiced. The term “exemplary” used throughout this disclosure means “serving as an example, instance, or illustration,” and should not necessarily be construed as preferred or advantageous over other embodiments presented in this disclosure. The detailed description includes specific details for the purpose of providing a thorough and complete disclosure that fully conveys the scope of the invention to those skilled in the art. However, the invention may be practiced without these specific details. In some instances, well-known structures and components may be shown in block diagram form, or omitted entirely, in order to avoid obscuring the various concepts presented throughout this disclosure. The various aspects of the present disclosure illustrated in the drawings may not be drawn to scale. Rather, the dimensions of the various features may be expanded or reduced for clarity. In addition, some of the drawings may be simplified for clarity. Thus, the drawings may not depict all of the components of a given apparatus or method.

In accordance with various aspects of the present disclosure, a welded orthopedic ankle support is provided. Instead of employing conventional stitching techniques, the materials that constitute the non-rigid body of the ankle support may be welded or thermally fused together to produce an ultra-low profile support. The welding of the various materials together enables otherwise intrusive seams and stitching lines to be substantially eliminated or reduced, which categories of items have caused substantial prior discomfort. This is in contrast to prior art solutions which are typically unnecessarily thick because they rely exclusively on stitching techniques to combine different regions of materials. Where multiple layers of material are used on the support, the problem is exacerbated because the profile of the support is simply further increased using these conventional solutions. In addition to excessively restricting movement of the foot/ankle system, higher profile supports render it difficult, if not impossible, to don ordinary footwear over the support.

In contrast, because the support materials are welded instead of sewn, the body of the ankle support has a naturally low profile with a compact volume that tends to be much smaller than existing solutions. Thermal fusion via welding produces an overall compression of the constituent materials. Further, using welding to assemble constituent parts of the body, it is generally easier for a designer to accurately design and achieve predictable and continuous properties over specific areas or regions of the ankle support. This is in part due to the fact that the belt behaves as a unitary segment with gradual changes in properties over different areas of the support, rather than as a collection of individual materials with substantially different properties and different degrees of directional freedom. Welding the materials causes the materials to become permanently affixed together across all welded regions, in contrast to the conventional approaches that rely purely on stitching or lamination.

Additionally, the ankle support according to the present disclosure tends to avoid sharp gradients in property transitions of materials in regions where those gradients are unnecessary. This benefit is due to the ability of thermal welding to integrate the fused materials together. This gradual transition of material properties, rather than sharp gradients in unnecessary regions of the support produced by conventional means, generally results in a body that has less discontinuities that are less noticeable to the user when the ankle support is worn. This, combined with a more compact and lower profile that makes it easier to don footwear over the support, results in an ankle support that is far more likely to be worn by a user as recommended by a specialist.

In accordance with other aspects of the present disclosure, the non-rigid bodies of the ankle support, that were traditionally adorned across most or all regions with uniform layers of high tension material to restrict foot and ankle movement, have been modified using concepts of anchors and tension segments to adopt a unique approach that combines efficacy in healing with comfort to the user. In an embodiment, the use of anchors and tension segments involves removing still further material and creating a still lower profile. The approach according to these aspects is premised on the inventors' recognition that certain major ligaments that govern action of the ankle joint terminate/originate in the calcaneus region of the foot/ankle system, or terminate/originate in a region proximate the calcaneus region. With this recognition, an ankle support as disclosed herein can be designed such that, in lieu of uniform regions of high tension, selectively shaped and oriented sections of a low-profile, tense material such as TPU can be used to effectively emulate action of these ligaments. In some embodiments, these high tension segments may originate in an area proximate the heel region and may extend across one or both sides of the foot/ankle system. For example, in an embodiment, an “anchor segment” of a low-profile, tense material may partially or wholly surround or otherwise encircle the open heel region of the non-rigid body. In alternative or additional configurations, the anchor segment may extend underneath a user's foot, e.g., under the sole or a portion thereof.

As shown further below, the emulation of the ligaments need not be direct such that the orientation of the applicable segments visibly correspond to the user's ligament anatomy. Rather, depending on the design and desired objectives, the emulation may be indirect. In this fashion, the restrictive segments of the ankle support need not directly resemble the orientation of corresponding ligaments, but in function, may operate as if they were substitutions for the ligaments. These embodiments are discussed further below.

The tension segments may be attached to the anchor segments and may run directionally or extend radially across one or both sides of the ankle/foot system to provide selective restriction of motion to the ankle/foot system. For example, in many instances of ankle injury it is desirable to avoid supination or pronation of the ankle to further exacerbate the injury. The anchor segment and tension segment(s) extending therefrom may be oriented and shaped to restrict supination or pronation of the foot or ankle. At the same time, and in contrast to conventional braces, the placement of the segments may be made to avoid interfering with normal forward and backward motion of the foot, i.e., dorsal and plantar flexion. The tension segments may effectively “substitute” for the ligaments in function while the ligaments heal.

In some embodiments, additional or alternative segments may be employed that do not necessarily originate at an anchor section proximate the heel or sole, but may instead extend laterally across the ankle/foot system. In these cases, the tension segments may extend indirectly from an anchor segment surrounding the open heel region via the use of the branching or fanning out of segments. In other cases, the tension segments can be oriented in a manner such that they are independent of the anchor section and can still perform their intended functions of inhibiting unwanted supination and pronation of the ankle. In still other embodiments, one or more tension segments may originate at an anchor section and extend across a region of the body to an attachment portion. An attachment portion may include an area on the body that is used to tighten and secure the body. In an embodiment, an attachment portion includes regions at respective, opposing ends of the body that use eyelets and laces, hook and loop, or other mechanisms to connect and secure the body to the foot/ankle system. In some embodiments, the attachment portions include thicker material to support the tightening of this portion of the body and also to receive tension segments from the sides of the body.

The anchor function may, but need not, be performed by discrete segments of material. Instead for example, the anchor segments may be implemented by the use of additional welding, stitching or binding of the support (e.g., proximate the heel or sole region) that provides the tension segment with a durable anchor.

The tension and/or anchor segments may be described for purposes of convenience and clarity as discrete segments of material. However, in practice, these segments may constitute any number of discrete pieces or sections of material. For example, while two tension segments may be apparent on a side of the body, the two tension segments may in practice be part of the same piece of material that was appropriately cut and shaped before the welding process. Similarly, the tension and anchor segments may in practice constitute a single integrated piece or section of material, or more than one piece or section of material. The anchor and tension segments may also, in some embodiments, use more than one layer of materials, or may use a composite material.

Some embodiments combine the benefits of welding with the selective use of the tension segments to produce an ultra-low-profile ankle support that may provide superior properties of comfort and efficacy in healing. Using an appropriate thermal fusion techniques as described below, different layers of material having different functions or objectives may be welded together to form an integrated, non-rigid body that performs these objectives at a minimum of thickness and with few, if any, interior seam lines or protrusions that the user may notice with tactile senses on the ankle and foot, and that may otherwise result in discomfort to the user. The discomfort in this event may significantly worsen over longer periods of time and particularly if a shoe or footwear is worn in conjunction with the support. The support of the present disclosure addresses these shortcomings, as shown in the non-exhaustive examples below.

The orthopedic support of the present disclosure is configured to inhibit ankle pronation and supination while promoting dorsi and plantar flexion. It should be noted that like any support, there will be some restriction on flexion at some point. In hyperflexion of the foot/ankle system, for example, there is a point where dorsi and plantar flexion is restricted in whole or in part. Thus, for purposes of this disclosure, when referring to the support permitting dorsi and plantar flexion, implicit in this and similar language are those cases where such flexion is minimally or marginally inhibited by virtue of wearing the support, and those cases of hyperflexion where dorsi and plantar flexion become inhibited.

The orthopedic support of the present disclosure is further configured, as noted above, to inhibit ankle pronation and supination. In addition to pronation and supination, there are also inversion and eversion events that occur and that are associated with the foot ankle/system. Injuries often occur as a combination of supination/pronation with inversion and/or eversion. Thus, for purposes of this disclosure, when referring to the support inhibiting or restricting ankle pronation or supination, implicit in this and similar language is the inhibition or restriction of inversion or eversion, alone or in combination with supination or pronation. Consequently, when the support is said to inhibit supination and pronation for purposes of this disclosure, it is also inhibiting inversion and eversion and therefore avoiding sustaining or exacerbating injuries associated with any of these events, whether alone or in combination. It is also noted that, for purposes of this disclosure, the reference to providing directional or tensile support to the ankle is synonymous with the identified selective restriction of the foot/ankle system to certain permissible movements (dorsi and plantar flexion) that do not inhibit healing, as discussed above.

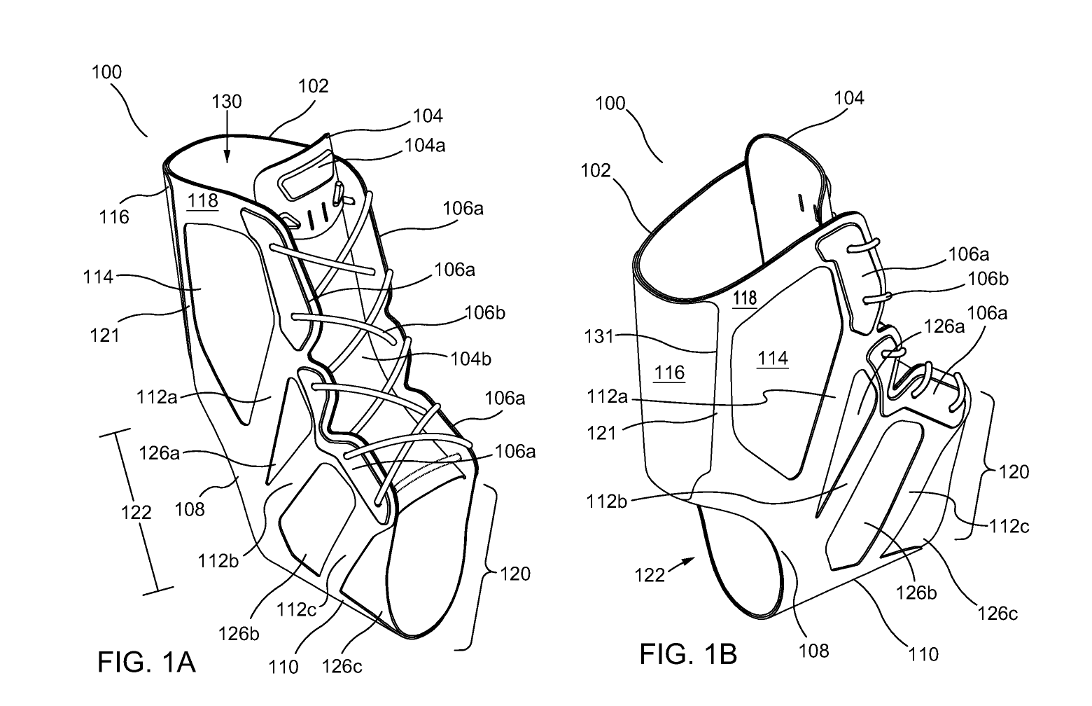

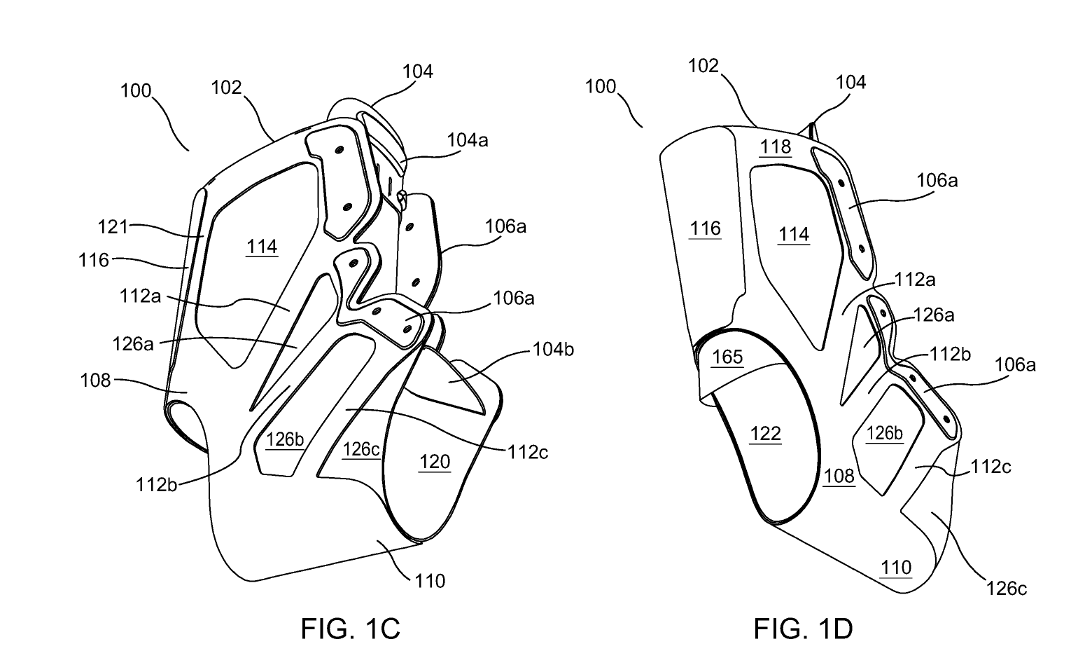

FIG. 1A is a front perspective view illustrating an example of an orthopedic ankle support 100 according to an exemplary embodiment of the present disclosure. FIG. 1B is a rear perspective view illustrating the orthopedic ankle support. Support 100 includes body 102, which represents the “shell” of the support. The body 102 in this embodiment is a bootie having an open-heel region 122 and open-toe region 120. Body 102 is generally non-rigid and therefore flexible. In other embodiments, certain structures attached to the body may be rigid. In general, the open-heel region 122 and the non-rigid nature of body 102 is intended to enable a user to don a shoe over the support; however, this need not be the case.

Body 102 includes flexible fabric regions 126a, 126b, and 126c. In an embodiment, regions 126a-c are porous and stretchable, enabling breathability and allowing for comfort. Regions 126a-c may be made very thin or may be relatively thin but also may include some padding for comfort. In a further embodiment, regions 126a-c constitute 3-D spacer mesh or a similar mesh material, although other materials may be equally suitable depending on the configuration of the support 100. The number and shape of regions 126a-c may also vary depending on the configuration, and three regions are shown by way of example only. Regions 126a-c may be included on one or both sides of the body 102.

Body 102 further includes sections 114. Although obscured from view in these figures, sections 114 may be present on both sides. In an exemplary embodiment, section 114 constitutes a porous, breathable fabric, similar to regions 126a-c. In another exemplary embodiment, sections 114 include a fastening material and are used as fasteners for a conventional figure eight structure, as shown further below. In the context of a fastener, sections 114 may include UBL or a similar material. In other embodiments not using a figure eight structure, sections 114 may be composed of a soft, breathable fabric as noted above. Alternatively, body 102 may be built shorter which may eliminate the necessity of a portion, or the entirety, of region 114.

While in the example of FIGS. 1A-B above regions 126a-c are stretchable and porous, body 102 also includes tension segments 112a, 112b, and 112c. In an embodiment, tension segments 112a-c may reside on only one side of the body 102. In a further embodiment, they may be included on both sides. Tension segments 112a-c, if on both sides, may vary in number and geometry on the different sides, or they may be identical. One or more tension segments 112a-c may be included in a particular ankle support. In contrast to the properties of regions 126a-c, tension segments 112a-c exhibit tension and lack flexibility and stretchability. In an exemplary embodiment, tension segments 112a-c are composed of thermoplastic polyurethane (TPU), although other materials may be equally suitable.

Shown bordering heel opening 122 is anchor tension segment 108. In an embodiment, anchor tension segment may at least partially surround heel opening 122. In another embodiment, anchor tension segment 108 may completely encircle heel opening 122. In still other embodiments, anchor tension segment 108 need not border heel opening 122 and may only be proximate heel opening 122 such that anchor tension segment 108 is not at the edge of the heel opening 122. Anchor tension segment 108 may additionally or alternatively reside below the foot. In the embodiment shown, anchor tension segment 108 encircles heel opening 122 (via region 116, discussed below). Anchor tension segment 108 may in some embodiments extend across a portion of the user's sole region to constitute anchor segment 110. The shape and orientation of anchor segment 108 and/or segment 110 may vary significantly from support to support without departing from the scope of the present disclosure.

Referring back to FIGS. 1A-B, anchor segment 108, with or without the extension segment 110, may act as a general support for the ankle/foot system and as such, the material(s) used may be tense and less stretchable, if at all. In an exemplary embodiment, anchor segment 108 and extension segment 110 may include relatively TPU. Anchor segment 108 and/or extension segment 110 may be used as an “anchor” to support tension segments 112a-c. That is, as shown, tension segments 112a-b may be attached to anchor segment 108 and tension segment 112c is attached to extension segment 110. Particularly because the areas that these segments occupy are below the shoeline, anchor segment 108, extension segment 110, and tension segments 112a-c may generally be made thin and are streamlined to avoid including unwanted protrusions or other tactile artifacts that may result in discomfort to a user donning the support. The geometry and number of these segments, and their presence on one or both sides of the support, may vary substantially depending on the desired objective and the nature of the injury.

In an alternative exemplary embodiment, anchor segment 108 is effected by stitching, binding or welding in lieu of the use of a discrete segment of material. For example, in this alternative embodiment, tension segment 112b may extend from a region proximate heel opening 122 radially upward along the side of the support, as shown. Instead of (or in addition to) anchor segment 108, tension segment 112b may be stitched, welded, or bound near heel opening 122 to provide the anchor function.

As is evident from the illustrations of FIGS. 1A-B, a user may don the support by slipping the foot through the upper surface 130 and through the support 100 until the heel comfortably protrudes through heel opening 122 and the toes extend from toe opening 120. The support is tightened via an attachment portion discussed below.

Body 102 further includes posterior region 116 of the support 100. In an exemplary embodiment, posterior region 116 is composed of a tense, supporting material such as TPU. The TPU or other material included in posterior region 116 may generally be thicker than the materials included in the above-referenced tension segments, primarily because the majority or entirety of posterior region 116 is above the shoeline and thus the added thickness should not result in discomfort to the user. In alternative exemplary embodiments, posterior region 116 is made significantly smaller or even eliminated. In the embodiment shown, posterior region 116 is used to provide support to the anchor tension segment and other portions of the body 102. The relative thickness of posterior region 116 also provides added strength and durability to the support 100, supports the posterior region of the user's foot, and assists in stabilizing the user's ankle along with the anchor and tension segments such that unwanted rotatory ankle motion is prevented.

In still other embodiments, posterior region 116 is thinner and is not used as a significant means of support, since the anchor and tension segments can be designed to sustain the pressures and forces that will be experienced over time by a mobile user of the support 100. In these embodiments, the support may also be made smaller to exclude a portion or substantially all of posterior region 116. Referring still to FIGS. 1A-B, support 100 includes an upper region 118 that is configured to surround a portion of a user's calf when the support 100 is worn. The material in this region may be designed to be relatively tense and thick due to its location above the shoeline. Upper region 118 may in an embodiment include TPU and may surround a posterior of the support 100 via posterior region 116. As demonstrated below, the body 102 may be constructed using multiple layers which may be integrated together. Thus, for example, upper region 118 may be integrated with posterior region 116 such that the two regions form a strong, albeit non-rigid body 102. The use of multiple materials integrated together, such as through welding, adds strength and durability to the support 100.

Referring still to FIGS. 1A-B, anchor tension segment 108 may be coupled to a vertical segment 121 which, at the top of the body 102, forms a junction with upper region 118 and posterior region 116. Vertical segment 121 may include TPU to provide support. The vertical line 131 (FIG. 1B) between vertical segment 121 and posterior region 116 is intended to demarcate the border between the regions and also, in the embodiment shown, describes the gradient of thickness, e.g., where the thickness of the body 102 changes quickly and visibly as a function of lateral position. In an exemplary embodiment, vertical segment 121 may include a tension segment similar to that of segment 112a; in other embodiments, vertical segment 121 may be integrated in the posterior region 116 or otherwise omitted.

The body 102 further includes an attachment portion 106a. One function of attachment portion 106a is to tighten and secure the support 100 to the user's foot/ankle system. In one embodiment, attachment portion includes thick sections of one or more materials that are durable and that have limited, if any, flexibility. For example, the thick regions of material 106a may include TPU. In the ankle support shown, there are four regions 106a that, along with lace set 106b, constitute the attachment portion. The attachment portion may be located proximate the respective vertical ends of body 102 and may include eyelets through which the laces 106b can be routed.

The attachment portion, while illustrated as a lace-up system such as those used in footwear, need not be so limited, and any suitable mechanism for securing the support 100 to the user's foot may alternatively be used. In other embodiments, the attachment mechanism may instead use a hook and loop closure system, e.g., with a D-ring, for tightening and attaching the body. Clasps or other structures may also be used alternatively or in addition to the eyelets.

The support 100 further includes a tongue 104. In an exemplary embodiment, the tongue 104 includes two portions. First, the tongue 104 includes a thin, stretchable material 104b coupled at its base to an interior portion of body 102. The tongue 104 may further include a thicker, more durable, and less flexible section 104a. The thickness of section 104a may be used, for example, to provide a cushion to a front of the user's foot when laces 106b are tightly bound. The material in portion 104b may, by contrast, be porous, breathable, and thin.

In an embodiment, ends of the tension segments 112a-c opposite anchor segment 108 and extension segment 110 are coupled to the attachment portion. For example, as shown in FIGS. 1A-B, tension segments 112a-c extend to regions of the body proximate attachment portion 106a. In this manner, tension segments 112a-c may be supported on one end via the anchor and extension segments 108 and 110, respectively, and on the other end via the thicker attachment portion protrusions 106a. It should be noted that, as shown in FIGS. 1A-B, tension segments 112a-c may or may not be directly connected to attachment portion 106a. Nonetheless, the thicker material included in attachment portion 106a advantageously may provide further support to the tension segments 112a-c. Thus, on one end, tension segments 112a-c are anchored by segments 108/110, and on the other end, tension segments 112a-c are supported by the attachment portion. Other embodiments not involving the use of the attachment portion for tension segment support, however, may be equally suitable.

Tension segments 112a-c may in other embodiments constitute a single visible segment extending across a side of the body 102. Tension segments 112a-c need not necessarily be coupled to the attachment portion as noted, since alternative orientations may be suitable in other implementations. The structure of body 102 advantageously enables a designer to orient the tension segments in any desirable way to achieve the intended objectives. As noted above, certain ligaments in the ankle extend from the subtalar joint or near that joint. In one embodiment, the support 100 capitalizes on the nature of major ligaments in the ankle extending to or from the calcaneus region or near the talo calcaneal region such that artificial, exoskeletal ligamentis tension segments on the support 100 can effectively emulate the function of the ligaments, regardless of whether the anatomical comparison is immediately visible or apparent. Thus, for example, where an injury involves a torn ligament in the ankle region near the calcaneus, one or more properly-oriented tension segments may act to prevent the ankle joint from improperly supinating or pronating, potentially with inversion or eversion (a function previously performed in part by the torn ligament) but without restricting all movement of the foot and ankle, as is the case with conventional braces.

In some embodiments, tension segments alone may be used without being attached to corresponding anchor segments or without being associated with anchor functions. Instead, in these exemplary embodiments, one or more tension segments may be disposed along one or both sides of the support and oriented or situated in a manner that assists in selectively stabilizing the ankle/foot system as described herein. Further, in other embodiments, tension segments may be coupled, connected or attached to an anchor segment/function underneath the fabric. That is, the connection may not be visible on the outside of the support, but may occur, for example, in connected layers underneath. For instance, the tension segment may disappear under a separate material but may be welded to other layers to cause a connection to the anchor segment.

In another aspect of the disclosure, the welding alone may alter the properties of the fabric to provide tensile strength that may serve the function of the tension segments, or anchor segments, or some combination of both. In an exemplary embodiment, a support may be manufactured by appending two or more pieces of stretchable material together to form the body. The materials may be joined at or near their edges via welding. Then, the resulting welded combination may be shaped to form the body of the support. Notably, in this embodiment, separate tension/anchor segments comprising a discrete material with high tensile strength (e.g., certain types, formulations or thicknesses of TPU or another suitable material) are not needed. Instead, the appropriate tensile strength may be present in the regions where the materials are welded. When oriented in an optimal direction, these regions effectively emulate the functions of the aforedescribed tension and/or anchor segments and provide the same directional support and selective restriction to the user with all the advantages of the low profile (if not providing even a lower profile) and the stretchability of the non-welded regions. The support according to this embodiment advantageously may be composed of fewer materials or one material and may provide an extremely low profile while maintaining the requisite selective stability for the ankle.

As noted above, conventional braces function by providing higher profile material with generally uniform tension across essentially the entirety of the foot/ankle system, and in many cases across the entire support. When tightened or secured onto the foot/ankle system, these supports assist in restricting unwanted rotatory movements of the ankle, but they also restrict natural movement that should not be and need not be restricted in most types of ankle injuries. These conventional braces, which provide tension all over the foot/ankle system, tend to restrict plantar and dorsi flexion as well, for example. That is, conventional braces restrict the natural forward and backward motion of the foot, which restriction is often not necessary for healing of the specific ankle injury, and particularly soft tissue injuries. By contrast, the support as disclosed in FIGS. 1A-B enables the designer to target the movement types to be restricted. This can be performed, for example, by orienting one or more tension segments to emulate the operation of ankle ligaments as described above. Also, the properties and behavior of the tension segments can be carefully controlled using welding. The resulting design can provide very predictable and precise results that are otherwise unavailable in solutions that use only stitching. Remaining portions of the foot/ankle system, such as foot portions corresponding to regions 126a-c, need not be covered with high tension material and may instead use the porous, thin and breathable material identified above to maximize the user's comfort. In other embodiments discussed above, the support may be composed of simple base materials welded together and shaped to form the body, wherein the welded edges are oriented in directions to correspond to high tensile regions, as discussed above. This approach may result in an ultra-low profile support that is extremely comfortable.

Welding is superior to conventional methods because when a weld is applied to even low tension or flexible material, directional tension will thereon be provided along the lines of the weld. That is to say, the weld produces permanent changes in the material's stretch characteristics. Accordingly, in an embodiment, the function of tension segments may be emulated along given weld regions or borders when applying thermal fusion along those regions/borders.

The fact that the user is able to flex the foot may enable the user to easily walk. Thus, the comfortable support according to the disclosure may provide the necessary motivation for the user to regularly don the support, which results in faster healing and a quicker path for the user to return to a mobile, productive state.

Edema.

The support 100 and variations described herein further reduce edema more effectively than conventional braces. The ankle is a more susceptible joint than other joints to prolonged injuries because it is located at a distance farthest from the heart. In ankle injuries, blood may amass in the injured area and cause substantial swelling in that area, which leads to pain. Conventional soft braces, which provide tight, often canvas-like materials that address edema through the non-uniform application of pressure against all of the tissue of the foot/ankle system, may cause considerable discomfort. By contrast, the support 100 according to the present disclosure may include regions of porous, stretchable material (e.g., regions 126a-c, lower tongue, etc.) restrained over the selected regions by nearby ligament-like segments, which are much more comfortable. Edema in the support described herein may be reduced by the comparatively even compression of the porous, soft, stretchable material against the tissue. This is in contrast to the uneven and irregular compression of tissue across a significant portion of, or substantially the entire, foot/ankle system, as provided by existing supports. The material in the present support may include spacer mesh or another suitable material with porous and/or stretchable properties, compressing the tissue substantially evenly through the bordering tension segments. The edema is therefore controlled in a manner that avoids causing additional discomfort to the user.

FIG. 1C is a side perspective view illustrating an example of an orthopedic ankle support 100 according to the present disclosure. FIG. 1D is a rear perspective view illustrating an example of an orthopedic ankle support according to the present disclosure. FIGS. 1C-1D offer different orientations of the support of FIGS. 1C-D, but omit the laces 106b of the attachment portion to add clarity to the illustrations. In addition, in the embodiment of FIG. 1D, a portion of cushion foam pad 165 is shown. Foam pad 165 may extend into the interior of the ankle support on the opposite of posterior region 116 for adding comfort and cushioning for the user. That foam pad layer 165 may be visible in some embodiments in an area near the heel region 122, as in FIG. 1D.

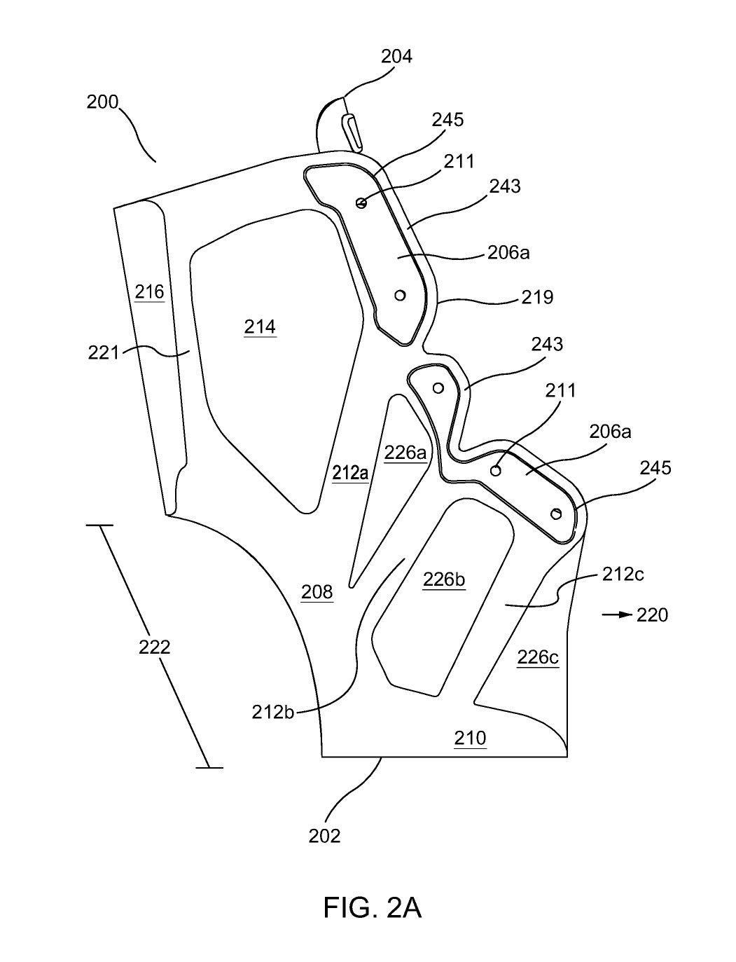

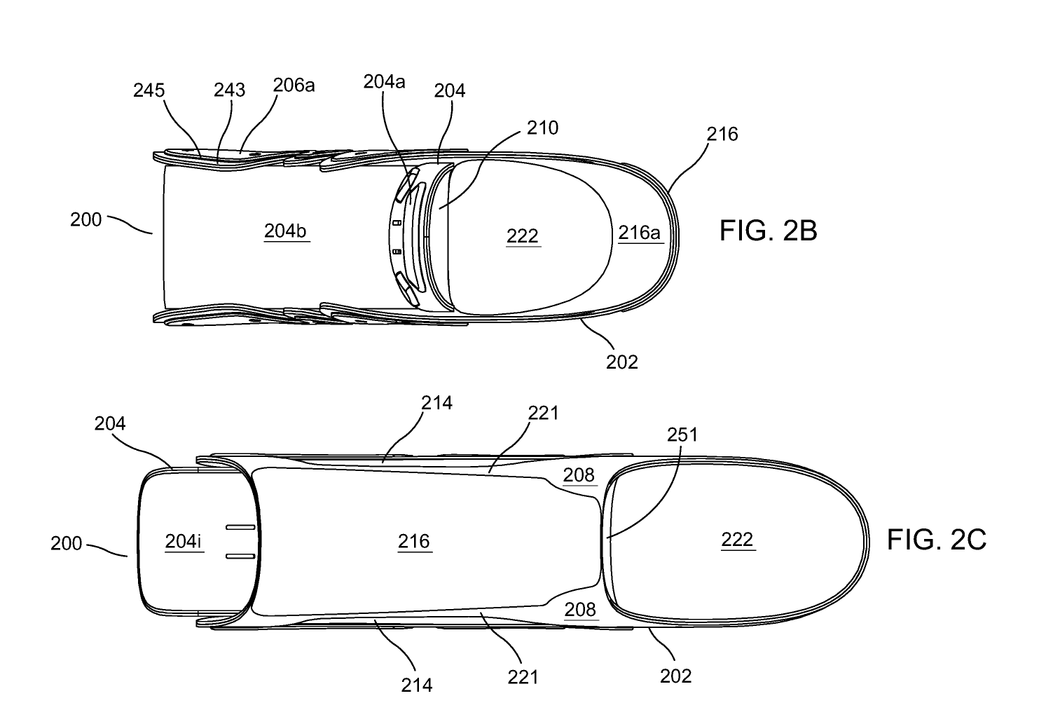

FIG. 2A is a side view illustrating the body 202 used in the orthopedic ankle support 200 according to the disclosure. Body 202 includes anchor segment 208 and extension segment 210. In this embodiment, anchor segment 208 borders heel region 222, although this need not be the case. Further, in this embodiment, anchor segment 208 extends around a circumference of heel region 222 for added support and stability. However, in other supports, anchor segment 208 may be more localized and may only, for example, extend partially around heel region. While this embodiment also utilizes a thin strip of material encompassing a portion of the sole region to constitute the extension segment 210, in other cases it may be preferable to omit material from the user's sole to ensure added comfort. Three regions 226a-c of porous spacer-mesh material are shown as before, although more or less regions may be available. Attachment portion 206a is also illustrated with an upper and lower segment of added thickness for strength and durability. Tension segments 212a and 212b extend from anchor segment 208 to the attachment portion 206a at an end 219 of the body 202 from a perspective of a front of the foot. Posterior region 216 is visible and adjacent a vertical segment 221, which assists in forming the exoskeletal structure of the body 202. Attachment portion 206a includes eyelets 211 through which laces can be routed. It is noted that region 243 may be considered in some embodiments to be part of the attachment portion. For instance, the borders 245 characterizing attachment portion 206a may represent visible thickness gradients, with region 243 having similar material but being thinner than the region bound by lines 245. Numerous embodiments of the attachment portion may generally be contemplated, including the use of rings and/or hook and loop attachment portions in lieu of (or in addition to) the lace-up mechanism shown in FIG. 2. The body 202 includes toe region through which the user's foot may protrude upon donning the support. Tongue 204 protrudes from an upper portion of body 202.

Generally, the heel region as described herein may cover different areas, and is not intended to be limited to a precise region. The heel region may vary from embodiment to embodiment provided it generally surrounds or approximately borders the calcaneus bone at the outer part of the heel. In some regions, therefore, the heel region may be designed to be very small, and the material of the support may encroach on the calcaneus. In other embodiments, the heel region may be designed to be much larger and therefore, for example, the heel opening on the support may take up much less material. In these latter embodiments, the fabric may be more distant from the calcaneus. The heel region for purposes of this disclosure is intended to include all of these embodiments and variations.

In alternative embodiments, the tension segments are designed as part of a wider section of material, rather than one or more strips. For example, in alternative embodiments, tension segments 212a-c may be combined into a single, wider strip or a pair thereof. Generally, the geometry of the tension segments can vary considerably while achieving the same result of selectively restricting motion using a low profile bootie design.

FIG. 2B is a top view illustrating the bootie 202 of FIG. 2A. The tongue 204 includes upper portion 204a and lower portion 204b. In an embodiment, the upper portion 204a is a comparatively thick, strong material and the lower portion 204b is a thin, flexible fabric. Attachment portion 206a along with its thickness gradient 245 and end region 243 are visible in FIG. 2B. From this perspective, a small portion of an interior of extension segment 210, which extends partially across the sole of the foot, is also visible. The region labeled 210 is also the region in which the user inserts the foot when donning the support 200. Heel opening 222 can be seen from this top view, as well as the interior region 216a of posterior region 216 (FIGS. 1A-D). The interior region 216a is the region against which the back of the ankle is flush when the support is worn.

FIG. 2C is a bottom view illustrating the body 204 of FIG. 2A. The heel 222 is directly visible from this view, along with the anchor segment 208 proximate the border of the heel. In addition, a portion of the vertical segment 221 and a small portion of the attachment section 214, used for attaching a figure eight structure (not shown), is visible. Region 216 represents the exterior of the posterior region. The tongue 204 is also visible along with a portion of the interior 204i of the tongue.

Tension and Anchor Functions.

The segments or structures that in effect, emulate the ligaments to selectively stabilize the ankle may, but need not necessarily, physically resemble the anatomical structure of the ankle ligaments. The examples below show that in some embodiments, more sophisticated, indirect means may be used to emulate the subject ligaments to selectively stabilize the ankle. Thus, staggered patterns of segments or other patterns that do not directly resemble how ligaments are routed may nonetheless effectively perform the functions of emulating major ligaments to achieve the intended objective. For purposes of this disclosure, a very low profile, ultra-comfortable orthopedic support can be achieved by welding together, on one or both sides of the body, one, two or more, or a network of segments (however shaped) to accomplish the directive of addressing the ankle injury at issue.

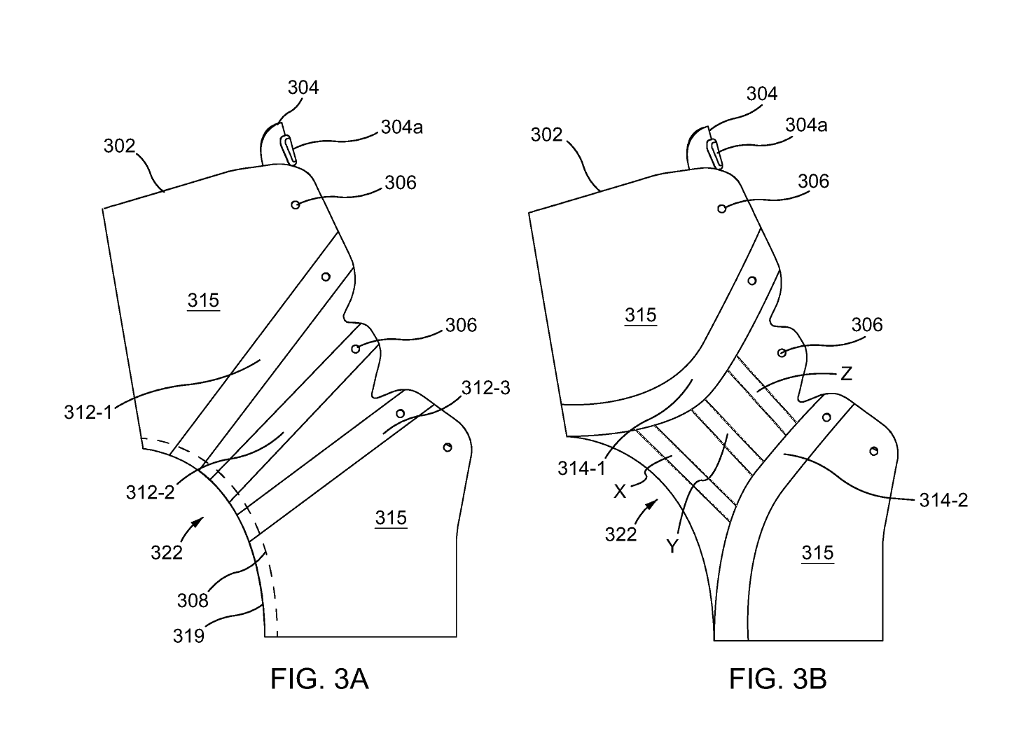

FIGS. 3A-B is a side view illustrating two exemplary embodiments of the tension segments of an orthopedic support of the present disclosure. It should be noted that the structures shown in the figure may apply to one or both sides of the body 302. One consideration for determining which side is whether the particular support is intended for a left foot, a right foot, or either. In other embodiments, both sides may include custom-designed tension segments to meet special requirements or to address a specific injury.

Referring first to FIG. 3A, body 302 may include a porous, non-rigid, soft and breathable material for comfort. In alternative embodiments such as that of FIG. 1A, additional regions for accommodating figure eight attachments or other structures may be included. FIG. 3A shows three segments of tense material 312-1, 312-2, and 312-3 extending from the heel region to the attachment portion, the latter shown for clarity as a set of eyelets. For the purposes of this disclosure, the three segments 312-1, 312-2 and 312-3 may serve as both tension segments and anchor segments. That is, the three segments are oriented across at least a portion of the body 302 and function to selectively inhibit ankle rotation. In addition, they are welded to the border 319 of heel region 322. Thus, in this embodiment, segments 312-1, 312-2 and 312-3 are anchored at the heel region. It should be noted that the segments need not be at the edge 319 of the heel region 322. Rather, for purposes of this disclosure, it is sufficient that the segments are generally proximate the heel region 322 or in other embodiments, the sole or lower part of the foot.

In addition, FIG. 3A includes dashed line 308. Line 308 may represent anchoring a segment via stitching, stitching binding, or an added welding step. Thus, the anchor segment may include stitching. In other exemplary embodiments, line 308 may represent an extra layer of adhesive material welded to secure segments 312-1, 312-2 and 312-3. Accordingly, the extra layer 308 may be considered an anchor segment, alone or in conjunction with the three segments 312-1, 312-2 and 312-3, because layer 308 includes an internal structure designed to provide additional security to the existing tension segments. It is also noteworthy that, while the tension segments 312-1, 312-2 and 312-3 extend to the attachment portion in this example and in fact extend to the edge of the body 302, this need not be the case. For example, in some exemplary embodiments, tension segments may be secured in an area short of the attachment portion, provided that they serve their function of restricting the appropriate type of motion—namely, improper supination or pronation of the ankle.

FIG. 3B shows an alternative exemplary embodiment of body 302 including material region 315 and attachment portion shown by eyelets 306 as before. In this embodiment, segments 314-1 and 314-2 may serve as anchors because they border the heel region. In one embodiment, segments 314-1 and 314-2 encircle the heel region, which may provide a significant amount of strength to ensure the support maintains the ankle in the correct position. Segments 314-1 and 314-2 may also be tension segments due to their functional tenseness and their purpose is to selectively restrict ankle motion as discussed above. Segments X, Y and Z represent tension segments that connect and further secure segments 314-1 and 314-2. In this example of tension and anchor segments, the segments may act in concert to provide the selective restriction in ankle mobility. Thus, the principles of the present disclosure allow for significant flexibility in designing a body that is particular to a type of ankle injury. In still other embodiments, networks of segments serving tension and anchor purposes may be constructed, and then thermally fused together to maintain the desired low profile.

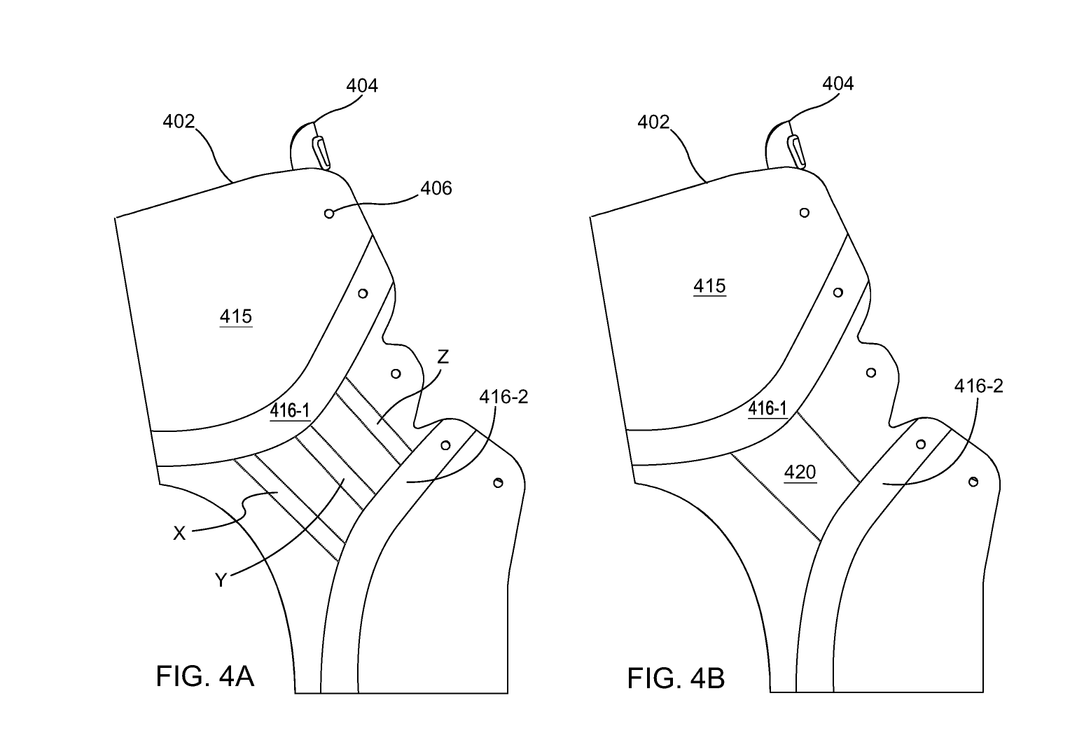

FIG. 4A is a side view illustrating another exemplary embodiment of the tension segments of an orthopedic support of the present disclosure. FIG. 4A, which includes body 402, tongue 404, attachment portion including eyelet set 406, material regions 415, and segments 416-1, 416-2, and X, Y, and Z, is similar to the embodiment in FIG. 3B except in the current embodiment of FIG. 4A, segments 416-1 and 416-2 do not directly border the heel region. Nevertheless, segments 416-1 and 416-2 sufficiently serve an anchor function as long as they are structurally adapted to provide an anchor, for themselves and, in this embodiment, for remaining segments X, Y and X. This may be accomplished through welding, stitching, binding, etc. FIG. 4B is similar to the embodiment of FIG. 4A, except that FIG. 4B is configured with one wider tension segment 420 instead of the three tension segments X, Y and Z of FIG. 4A.

In some alternative embodiments, less higher tension material may be used provided it is sufficient to perform the anchor and tension functions for inhibition of selective motion. Generally, when less area is used for tension and anchoring, more area may instead be provided as soft, breathable fabric, maximizing the user's comfort. Further, while a number of the embodiments discussed above reference segments in the context of rectangular-based shapes, the disclosure is not so limited and the segments may take on any shape desirable for performing the necessary functions. This may include, for example, use of multiple segments that are very thin and thus each have a very small surface area. This may also include one larger segment, and/or oddly shaped segments. Also, as was seen with reference to FIG. 3A with the internal layer 308, the segments need not necessarily be situated at the surface of the body.

In an embodiment, the support 200 is constructed without any stitching under the shoeline. Thus, anchoring is accomplished using solely welding techniques and appropriate materials. As discussed, the absence of stitching may result in an extremely thin support that is both highly effective and palatable to the eyes of an injured user.

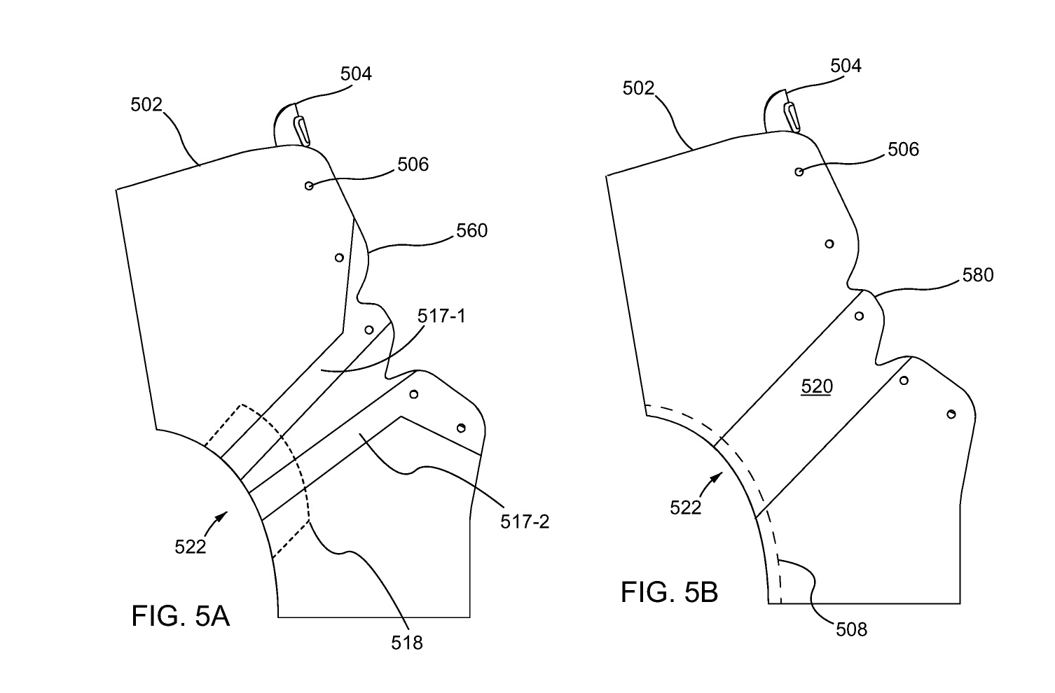

FIG. 5A is a side view illustrating another embodiment of the tension segments of the orthopedic support of the present disclosure. The body 502 of 5A includes tongue 504, eyelets 506, an anchor segment 518 which may include a piece of durable material welded proximate the heel region or directly onto a border of the heel region. In other embodiments discussed herein, the anchor segment may at least partially encompass or encircle the heel region 522 either at the edge of the heel region or adjacent the heel region 522. These embodiments may increase the overall strength of the support. Tension segments 517-1 and 517-2 are anchored by anchor segment 518 and extend radially out to the border 560 of the attachment portion.

FIG. 5B is a side view illustrating another embodiment of the tension segments of the orthopedic support of the present disclosure. This example is similar to the example of FIG. 5A except for the anchor/tension structure. Tension segment 520 (which also may include an anchoring function) extends to a border 580 of the body 502 where an attachment portion (including eyelet set 506) ordinarily resides. Instead of two tension segments as in FIG. 5A, a single tension segment 520 having a wider surface area to increase tension force is employed.

Referring still to FIG. 5B, dashed line 508 represents an added stitching or binding, or a welding step, to provide a further anchor segment for the tension segment 520. In an embodiment, the stitching, binding or welding 508 extends around the entire periphery of heel region 522 for added strength.

The embodiments in FIGS. 3A-B to FIGS. 5A-B have generally not included reference to an extension segment that may, for example, extend from the anchor proximate the heel to a region under the sole of the foot or a portion thereof. Such segments may be included in some embodiments (see, e.g., element 110 of FIGS. 1A-D) however, and may be added to the prior embodiments where desired. These extension segments may provide greater flexibility to the designer because it can act as an additional anchor segment for placing further tension segments that extend radially from a side of the foot. In an exemplary embodiment, the thickness profile for a material to be used under the foot is made extremely small to minimize or eliminate any discomfort experienced by the user resulting from the user's prolonged mobility.

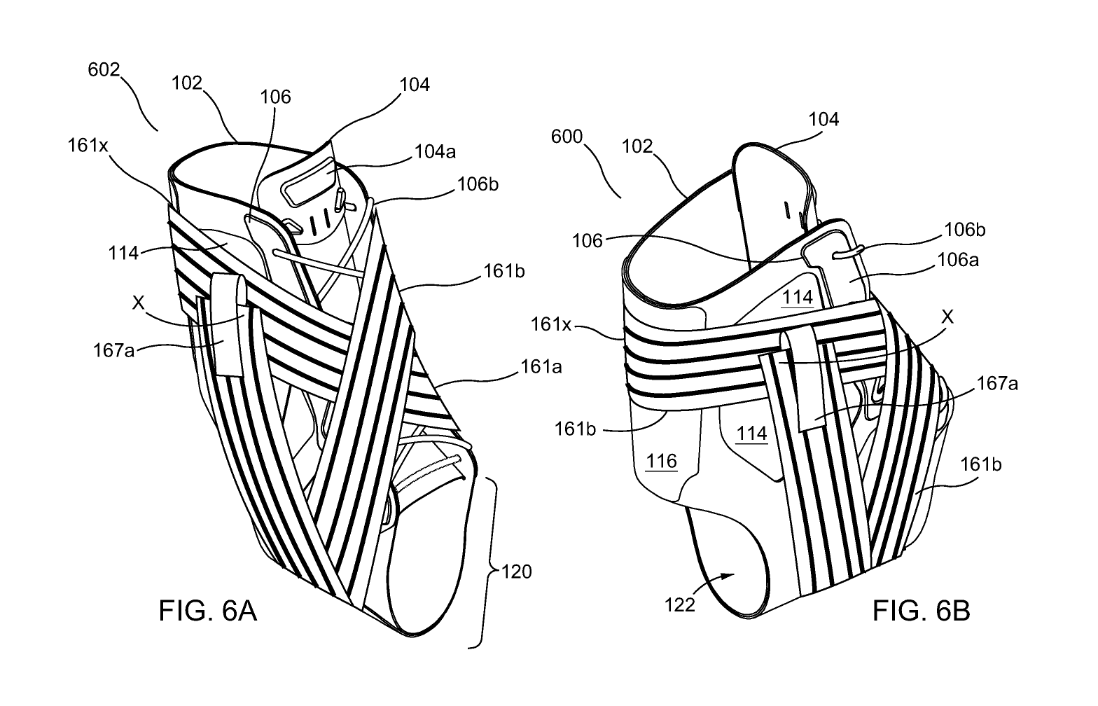

Figure Eight Structures. Figure Eight Structures can be Added to the Orthopedic Ankle Brace in an exemplary embodiment to provide further support and to add a layer of protection to restrict the ankle from sudden or unexpected supination/pronation. To this end, FIGS. 6A-B are side perspective views illustrating an embodiment of the orthopedic ankle support 600 with a figure eight structure attached. In an embodiment, a center of the figure eight structure 161x is stitched or welded onto a rear of posterior region 116. A function of the figure eight structure is to provide additional support for preventing rotatory motion, including supination and pronation, of an injured ankle without substantially interfering with dorsal and plantar flexion. In an embodiment, the figure eight structure may be composed of a thin and substantially inflexible material for wrapping around the body 102. After donning the support 600 and securing the support 600 using the attachment portion 106a-b, the user may wrap a first side 161a of the figure eight structure around and then under the body 102 as shown. The first side 161a of the figure eight structure may then terminate on a UBL section of region 114. A second side 161b of the figure eight structure may also wrap around the body 102 in the opposite direction and terminate on a UBL section of a corresponding region 114 on the other side of the body (obscured from view). The basic premise of function is that each of the first and second sides 161a-b of the figure eight strap wraps around the body 102 in opposite directions and then are securely fitted onto corresponding regions 114. These additional straps add resistance to any tendency of the ankle to twist or rotate improperly and represent an optional inclusion to the tension and anchor segments discussed herein.

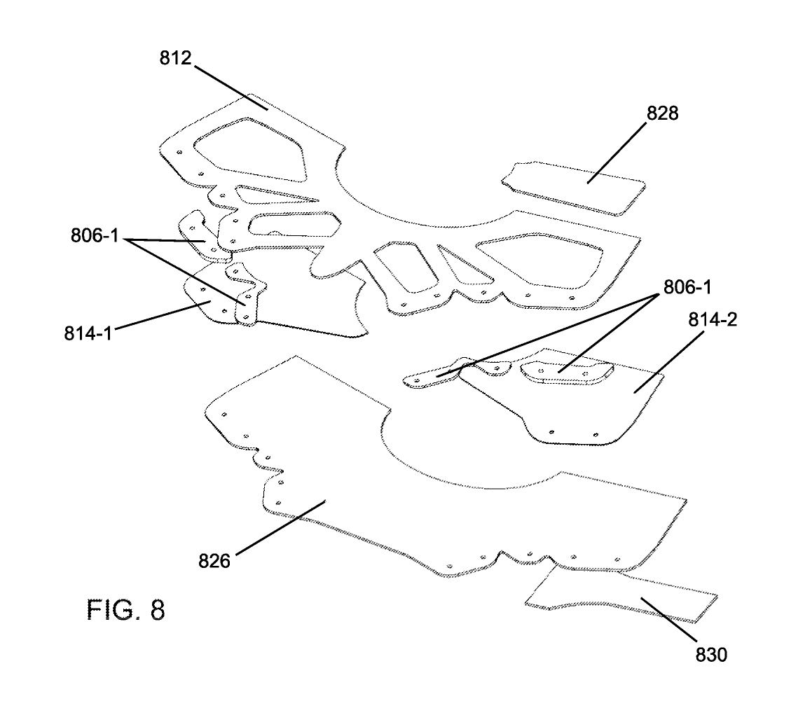

In an embodiment, the first side 161a includes a hook material that engages with the UBL material on the region 114. In addition, in some situations one side of the figure eight structure may interfere or overlap with the other side, such as at area x where the first side 161a of the figure eight structure terminates. Similarly, on the opposite side of the body 102 (obscured from view) a second side 161b of the figure eight structure may similarly interfere with the first side 161a. To alleviate this problem, the portions of the FIG. 8 structure that overlap (generally at area “x” with reference to one of the overlaps) contain additional hook and loop material for engaging with complementary hook and loop material 167a at the end of the first side 161a and at the end of the second side 161b (obscured from view). Thus, referring to FIG. 6B, the hook material of 167a comes into contact with both UBL region 114 and an extra patch of UBL on a portion of strap 167b (obscured from view). A similar contact mechanism may occur on the opposite side where an end of the second side of figure eight structure 161b meets region 114 on the opposite side. More generally, such interference situations may be addressed by placing additional attachment material (e.g., hook and loop material) where needed. The added thickness at this upper point of the support will likely not cause discomfort to the user since it is sufficiently above the foot/ankle system.

Elastomer Specifications.

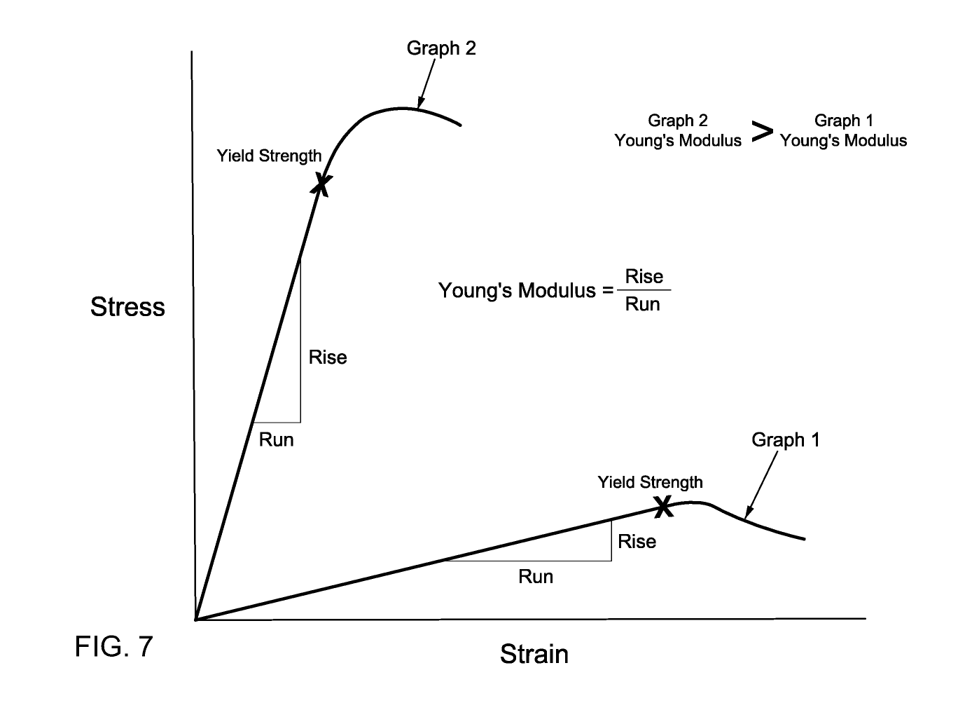

Certain commercially-available orthopedic braces are currently composed of a flexible body material layered together with an elastomer component to improve compression. These elastomers, however, usually have a low Young's modulus or a low modulus (i.e., low stress, high strain) of elasticity. This means when a stress is applied to the elastomer, the resistance force stays relatively constant while the strain value increases before permanent deformation or yield strength maximization. As evidenced by the stress-to-strain curve (see FIG. 7, Graph 1), these conventional orthopedic brace solutions are not ideal for primary use in many or most orthopedic ankle braces. For example, use of a low Young's modulus elastomer characteristic in these conventional braces is not useful for preventing such occurrences as an ankle “blow-out”, where the ankle is injured or the existing injury is suddenly exacerbated. Rather, the stress and strain curve that is ideal for an ankle brace has a high Young's modulus (i.e., high stress low strain) so that the stress to strain curve will be at a higher slope. Thus, for example, for stresses applied to the brace that cause a significantly small amount of material deformation, a large value of resistance to the applied stress increases accordingly. The high stress and low strain material is therefore more ideal for use in the ankle support of the present disclosure to reduce the impact of the ankle during an ankle blow-out.

FIG. 7 illustrates an exemplary pair of graphs of stress versus strain and the resulting value for Young's modulus for different materials. Graph 1 illustrates a material having a low modulus of elasticity (low stress high strain). The material associated with Graph 1 has a low yield strength, so the Young's modulus which is the slope=rise/run, is low. Where the applied stress value has a marginal increase, the resulting strain value has a high increase. These properties mean that the subject material of Graph 1 cannot properly function as a primary material in an ankle brace because the material has primarily elastic properties. Namely, the material will elongate even upon application of a low force, and the ankle will roll out while the material elongates. For this reason, commercial orthopedic braces that use neoprene, soft silicone or another elastic material as a means of resistance are generally used only to reduce swelling by providing elastic compression and cannot be used to restrict ankle supination/pronation in the manner disclosed by the orthopedic braces described in the aspects herein.

Referring still to FIG. 7, Graph 2 illustrates the behavior of a material having a high yield strength as stress is applied. As shown, Graph 2 has a high slope, which means that the Young's modulus is higher. Accordingly, when force is applied to the material, the stress increases while the elongation increases at a slower rate than graph 1. That is, the strain of this material increases only marginally as a function of an applied stress. Accordingly, in an exemplary embodiment, the tension and anchor segments of the present disclosure use a material that more closely resembles the behavior of a material described by Graph 2. As these graphs illustrate, a high modulus of elasticity is synonymous with a high Young's modulus. Thus, the anchor and tension segments may be configured to include these properties in order to provide the requisite directional support resulting in the desired selective restriction of movement in the ankle/foot system. It should be noted that other portions of the support may also exhibit high tensile strengths to accomplish other, sometimes unrelated objectives, and that these properties of high Young's modulus need not be limited to tension segments. As one example, two stretchable materials (which may be the same material) that are welded together may produce a resulting border region with high tension, as discussed above.

An ankle blow-out usually occurs as a result of a very quick and explosive motion. The inventors have observed studies that show that explosive inversion or eversion can happen in as low 40 milliseconds, or quicker than the blink of an eye. Meanwhile, the user's reaction to the ankle blow-out is 50 milliseconds, or longer. See, e.g., Daniel T P Fong et al., Understanding Acute Ankle Ligamentous Sprain Injury in Sports, Jul. 30, 2009 (https://www.ncbi.nlm.nih.gov/pmc/articles/PMC2724472/.). Accordingly, one or more materials may be used that can properly respond to such a fast event, such as the welded materials described above.

In an embodiment, the tension and anchor segments are composed of TPU. However, the disclosure is not so limited, and other materials having similar modulus of elasticity may be used. While the exemplary elastomers used herein include the TPU tension segments as described and configured herein, other materials or composites that, when welded, yield a sufficiently high modulus of elasticity may also be possible. Based on reference test data, elastomers used in exemplary embodiments of the present disclosure may have a combined modulus of elasticity higher than 380 PSI (pounds per square inch) or 2.8 MPa (Megapascals) (ref test data). Elastomers with a low modulus of elasticity, such as “rubber band” like materials, generally have a modulus of elasticity of up to around a maximum of 120 PSI or 800 Pa and as such, are unsuitable for primary use as tension or anchor segments in the embodiments disclosed herein. It should be understood, however, that such materials with a low modulus of elasticity may be appropriately used in other portions of the support, such as the body (including regions 126a-c), the lower portion of tongue 104b, etc. (see FIG. 1A).

Flexural modulus vs tensile strength may also be properties to consider in determining which materials can be used for the ankle support disclosed herein. Generally, for the ankle support to be comfortable, the materials should be somewhat flexible. This flexibility, which may help conform the support to the user's foot shape and may generate a more uniform compression. In an embodiment, the materials have a relatively low flexural modulus to conform to the user's foot shape but a high tensile strength to prevent the ankle rolling or rotating in tension. In a further exemplary embodiment, the ankle support as disclosed herein takes advantage of the tensile direction to prevent the ankle inversion or eversion. Thus, for example, the anchor and tension network as disclosed herein may include TPU originating from one side of the foot and running along a periphery of the calcaneus to the other side of the foot (e.g., segment 108, FIGS. 1A-B) along with the attachment portion 106a (FIG. 1A) in the front of the foot and the tension segment(s) (e.g., 112a-c, FIG. 1A) therebetween. This radial direction is the tensile direction that prevents the rolling of the ankle. Unlike stirrups and many injection molded plastic which high flexural modulus and tensile strength, this embodiment has low flexural modulus while still maintaining high tensile strength.

Another property that can affect the elasticity of an elastomer is temperature. Generally elastomers that are thermoset have a lower resistance to temperature. This means that the elastomer at issue may have different properties when the temperature is at the glass transition of the polymer. For example, TPU and similar materials are thermoplastic, and as a result, have a higher resistance to temperature. These types of materials may be more suitable for primary use in the ankle support so that temperature variations will not significantly affect the elasticity of the support. Temperature differences may become significant in a variety of situations. For example, where an ankle support is worn by athletes, temperature can increase dramatically either by means of their activity alone, or by friction against the shoe when the athlete performs when wearing the ankle support along with regular shoes.

Temperature resistance may also be relevant to abrasion of a material. Abrasion of thermoset materials is generally low. More specifically, because of their low temperature resistance, the materials are easy to break down when glass transition of the polymer is reached. To account for abrasion resistance for the ankle support, the temperature resistance can be made higher by selecting materials with a corresponding high temperature resistance.

It is also noteworthy that ankle supports are designed to restrict ankle motion. Viewing the action of the ankle support from an energy conservation perspective, the kinetic energy transfers into elastic potential energy. TPU, which may be used in several regions in various embodiments (e.g., elements/regions 104, 106a, 108, 110, 112a-c, 116, 118, 121, FIGS. 1A-B) generally can absorb more kinetic energy without significant resulting movement, which can, in operation, prevent ankle from blowing out more.

It will be appreciated that, although TPU may be used in various embodiments, the disclosure is not limited to TPU, and a number of other materials may be equally suitable without departing from the spirit or scope of the disclosure. In addition, while the use of elastomers with specific properties has been discussed, the ankle support as disclosed herein need not be limited to any specific elastomer. Further, although other elastomers may not be suitable for use in certain aspects of the present ankle support, the elastomers may be appropriate for use in other portions of the support or for other purposes. In addition, various composites may be suitable for use in the ankle support that have not been specifically identified. Thus, the previous discussion is not intended to be limiting, but rather is meant to identify properties of materials that may be relevant to various embodiments.

FIG. 8 is a perspective exploded view illustrating an exemplary layering of materials for constructing the body of the orthopedic ankle support. As a first step in an exemplary method of constructing the body, a designer selects the suitable materials and their individual dimensions. The designer then may have the materials cut manually or using an automated cutting mechanism. Referring to the exploded view of FIG. 8 from the bottom up, the first layer 830 may include a segment of posterior spacer that will eventually be used in joining the heel opening together. The second layer 826 may encompass the layer of spacer material or another stretchable, porous fabric. As noted, the spacer layer in this embodiment will be distributed throughout the entire body of the support. Later, after the welding of the materials, the spacer layer may contribute and integrate beneficial properties of flexibility and comfort into other layers.

Above the spacer layer, optionally, another layer (not shown) may be included in some exemplary embodiments for providing plastic stirrups to the body. The layer may include, for example, a hot melt board cut to implement a stirrup on each side. After welding, the support may thereupon incorporate stirrups on each side. A pocket may be added to accept another layer. The additional layer can be added internally or externally. The stirrups may essentially provide an added layer of stability and support on each side of the leg. Unlike traditional rigid hard plastic stirrups that are built onto the sides of the support after the support is assembled, which creates additional unnecessary size to the support, the plastic stirrups as disclosed herein are much more compact because they are welded into the body during assembly. The shape of a plastic stirrup layer according to an embodiment is essentially two rectangles connected together by a smaller strip, which may be curved. In other embodiments, the plastic stirrups are omitted.

A third layer above layer 826 (or above the plastic stirrup layer, where applicable) may include two symmetrically distributed regions 814-1 and 814-2 that correspond respectively to regions 114 (FIGS. 1A-D) on each side of the body. In an embodiment, these regions include UBL, or a hook material or other connecting material. As discussed, these regions may be used to secure figure eight structures. In other embodiments not incorporating figure eight structures, these regions may be omitted and only the spacer material 826 will be present in regions 114 in the end product.

A fourth layer may include hot melt eyelets for adding thickness and support to the attachment portions 806-1 and 806-2. The attachment portions 801-1 and 806-2 may be constructed to include eyelets, and may add thickness to add further support to this region. It should be noted that because each layer will be incorporated into the final product, in the mold, the eyelets will be included in every layer that intersects with the attachment portion. In an embodiment, the eyelets may be used in some hardware equipment to align the various layers. The resulting attachment portions may include TPU (see below), the welded hot melt eyelets 806-1 and 806-2 for added support, and spacer layer 826.

A fifth layer may include the tension and anchor segments. In this embodiment, the tension and anchor segments are uniformly composed of TPU and represent in principle one piece of material. As discussed with reference to previous embodiments, this need not necessarily be the case, and in some embodiments additional, alternative, or different segments or pieces of TPU or other material may be combined or integrated to form the final product. Moreover, upon welding, the TPU will be integrated with the materials in the other applicable layers such that the properties of this portion of the body will vary gradually over the surface area of the body. Nonetheless, the TPU may provide the necessary tension to stabilize the supination/pronation of the ankle as described. Here again, materials other than TPU may be equally suitable for use in this layer.

A sixth layer may include a segment 828 of posterior TPU to assist in joining the heel portion. In practice, more or less materials and/or layers may be used. In addition, in some embodiments, a single layer may be populated with more than one material, although care in these cases should be taken to ensure that the dimensions and layers align properly.

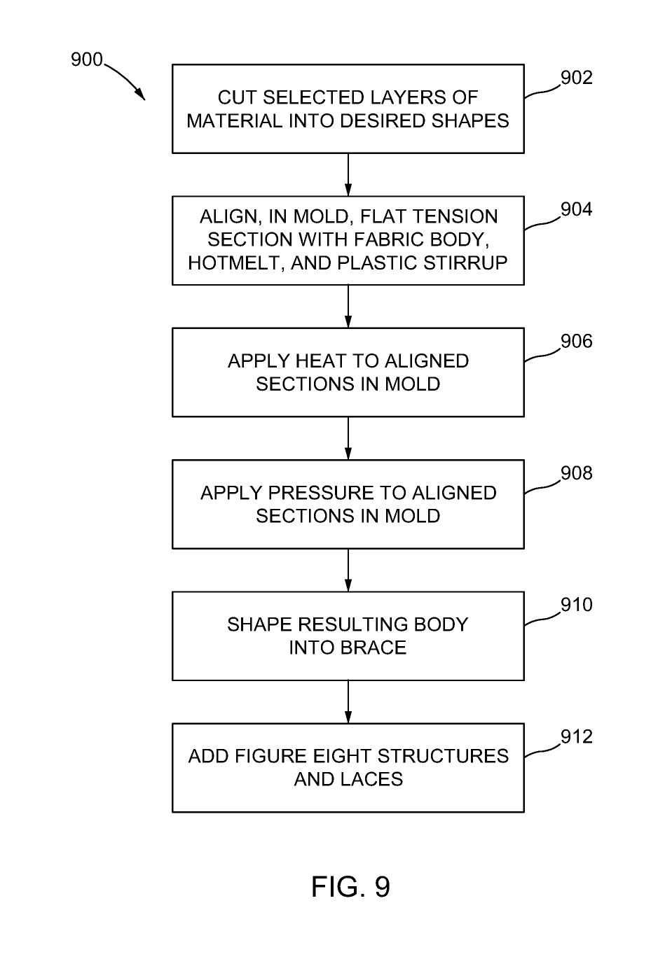

FIG. 9 is a flow diagram illustrating an exemplary method 900 for producing an orthopedic ankle support according to the disclosure. The selected layers may be cut into the desired geometrical shapes (902). This procedure may be manual or, for volume production and to achieve precision, it may be automated via appropriately programmed cutting machinery. The selected layers are then placed in an appropriate mold, where they are aligned vertically and horizontally in the correct position (904). Examples of the layers were discussed with reference to FIG. 8 and include the flat tension section (for the anchor and tension segment(s)), hot melt boards (e.g., for reinforcing attachment portion and eyelets, etc.), and in some embodiments, the plastic stirrups. Other layers were previously referenced, and still others may be desirable depending on the objective. For example, in some embodiments, separate layers for introducing different materials into the anchor and tension segments may be used. Alternatively, the tension section may be maintained flat, but more than one piece of material may be used in that layer.

Thereupon, the mold may be closed to create a closed environment for the aligned layers and to create pressure on the layers, and the welding process may commence. It is anticipated that for purposes of the present disclosure, any number of welding and thermal fusion processes may be used to produce the body. Caution must be taken prior to commencement of the welding, however, to properly configure the layers and welding tool such that the more delicate regions of the body (e.g., the UBL and spacer regions such as 114 and 126a-c (FIG. 1A)) are not welded or otherwise damaged by the significant heat that will be applied to weld the other layers. Conventional techniques are available to address this concern.

Welding is a known fabrication process involving the use of heat and pressure on thermoplastics and other materials to integrate layers of materials together. Unlike stitching or other connection means, where the layers remain substantially independent of one another and retain their own chemical properties, welding typically integrates the layers together as one layer with a new set of properties. Where the materials and welding parameters are properly selected, the new set of properties is typically superior to the old set of individual properties of the constituent layers.

Heat is applied to the aligned sections in the mold to integrate the material layers (906). Welding also involves pressure, such that sometimes substantial amounts of pressure are applied to the aligned sections. The application of heat and pressure may be concurrent, in part or in whole, or may be in discrete steps, depending on the welding process employed. Accordingly, a separate step of applying pressure to the aligned sections is identified in FIG. 9 (908).

After the welding process, what remains is a unitary body in a flat position. Using an artificial foot/ankle system or other shaping means, the body is curved and shaped into the position of a support. Additional steps may be undertaken in the fabrication process to maintain a permanent curve to the body; these principles will be understood by those skilled in the art upon reviewing this disclosure. A posterior portion (116) can at this point be welded onto respective ends of the curved body to finish the formed body of the support. Thereafter, any additional elements may be added onto the support. For example, the figure eight structures may be stitched (or welded) onto a posterior region of the body as previously discussed. Where the attachment portion is a lace-up configuration, laces may be provided through the eyelets.

While spacer mesh material and other specific materials have been discussed in the context of various embodiments throughout this disclosure, it should again be emphasized that the support as contemplated herein needn't be limited to or require these materials. In other embodiments, materials having similar or superior properties may be substituted for the exemplary materials described while maintaining adherence to the principles of the present disclosure.

Depending on the nature of the injury and the size of the user's foot, numerous different types of supports may be manufactured using the above-described procedures. Custom supports may also be produce to address unique injuries or to meet unique needs, e.g., the needs of an athlete, and to accommodate users with different expected levels of activity, etc. Over time, additional iterations of ideal configurations of the tension segments may become preferred, with one objective to maximize the amount of comfortable fabric layers and potentially minimize the area consumed by the anchor and tension segments.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

The various aspects of a flexible support presented throughout this disclosure are provided to enable one of ordinary skill in the art to practice the present invention. Various modifications to aspects presented throughout this disclosure will be readily apparent to those skilled in the art, and the concepts disclosed herein may be extended to other flexible supports. Thus, the claims are not intended to be limited to the various aspects of this disclosure, but are to be accorded the full scope consistent with the language of the claims. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

1.-36. (canceled)

37. An ultra-low profile ankle brace, comprising:
a body comprising a shell including an open heel region and an open toe region, the body formed of a flexible, stretchable material;
a tongue connected to the body;
a reinforcement layer welded, without stitching, to the body, the reinforcement layer having a lesser flexibility and a lesser elasticity than the body, and comprising:
an anchor tension segment extending to and at least partially enclosing the open heel region;
an extension tension segment extending proximally from the anchor tension segment along a plantar region of the support;
a vertical tension segment extending from the anchor tension segment in a vertical orientation; and
a plurality of elongate ligament directional tension segments extending from the anchor tension segment in respective radial directions; and
an upper region connecting a first end the vertical tension segment to a first end of selected elongate ligament directional tension segment; and
a planar attachment member welded to the upper region of the reinforcement layer;
wherein the reinforcement layer forms windows than circumscribe and expose regions of the body to form expansion zones between the plurality of elongate ligament directional tension segments, the expansion zones entirely bounded by the reinforcement layer.

38. The ultra-low profile ankle brace of claim 37, wherein the expansion zones are adapted to apply an even compression to adjacent tissue via tension in bordering tensioning segments.

39. The ultra-low profile ankle brace of claim 37, further comprising a foam heel pad at an interior surface.

40. The ultra-low profile ankle brace of claim 37, wherein the planar attachment member, the reinforcing layer, and the body are welded simultaneously.

41. The ultra-low profile ankle brace of claim 37, further comprising a posterior support welded to the anchor tensioning segment and the vertical tensioning segment, the posterior support extending from an upper surface of the brace to the open heel region.

42. The ultra-low profile ankle brace of claim 41, wherein the posterior support has a greater thickness than a thickness of the reinforcing layer.

43. The ultra-low profile ankle brace of claim 37, wherein the reinforcing layer is welded to an inner surface of the body and is not visible when the brace is worn by a user.
